(19) United States
(12) Patent Application Publication
(10) Pub. No.: US 2016/0120974 A1
(43) Pub. Date: May 5, 2016

(54) SEMI-LIVE RESPIRATORY SYNCYTIAL VIRUS VACCINE

(71) Applicant: AMVAC AG, Zug (CH)

(72) Inventors: Marian WIEGAND, Muenchen (DE); Christine KAUFMANN, Muenchen (DE)

(73) Assignee: AMVAC AG, Zug (CH)

(21) Appl. No.: 14/896,866

(22) PCT Filed: Jun. 10, 2014
(86) PCT No.: PCT/EP2014/001575

§ 371 Date: Dec. 8, 2015

(30) Foreign Application Priority Data
Jun. 10, 2013 (EP) 13 002 973.9

(51) Int. Cl. A61K 39/155; C12N 15/86; C12N 7/00

(52) U.S. Cl. CPC A61K 39/155; C12N 7/00; C12N 15/86; A61K 2039/5256

(57) ABSTRACT

The present invention relates to a semi-live respiratory syncytial virus (RSV) vaccine, which comprises a genome replication-deficient Sendai virus (SeV) vector expressing a chimeric RSV/SeV F protein. Furthermore, the present invention relates to a method for the production of the genome replication-deficient SeV vector of the present invention, and the use thereof in the treatment of RSV infections and RSV infection-related diseases.

### FIELD OF THE INVENTION

The present invention relates to a semi-live respiratory syncytial virus (RSV) vaccine, which comprises a genome replication-deficient Sendai virus (SeV) vector expressing a chimeric RSV/SeV F protein. Furthermore, the present invention relates to a method for the production of the genome replication-deficient SeV vector of the present invention, and the use thereof in the treatment of RSV infections and RSV infection-related diseases.

### BACKGROUND OF THE INVENTION

Many of the viral vaccines used today, including those of measles and some influenza vaccines, are based on attenuated viruses and generate good and long-lasting prophylactic humoral and cellular immune responses (Amanna et al., N. Engl. J. Med. 357:1903-1915, 2007). Such live attenuated vaccines are created by reducing the virulence of the used virus, but still keeping it viable (or “alive”).

However, safety of live vaccines is constantly being discussed as they have also been associated with genetic instability and residual virulence (Ehrenfeld et al., Expert. Rev. Vaccines 8:899-905, 2009). Possible reversion of attenuating mutations, as seen with the Sabin polio vaccine (Salk, D. and Salk, J., Vaccine 2:59-74, 1984; Kew et al., Annu. Rev. Microbiol. 59:587-635, 2005), or finding the right balance of attenuation, which complicates for instance the development of live attenuated respiratory syncytial virus (RSV) vaccines (Luongo et al., Vaccines 27:5667-5676, 2009), exemplify the shortcomings of live vaccines.

Given the limitations present in using live vaccines, viral vectors have emerged as potent and defined approaches with immunogenic characteristics similar to live attenuated vaccines (Abdulhaqq et al., Immunol. Res. 42:219-232, 2008; Liniger et al., Vaccine 27:3299-3305, 2009; Zhan et al., Vaccine 26:3480-3488, 2008; Slobod et al., Vaccine 22:3182-3186, 2004). However, live attenuated viral vectors often face similar safety concerns as the long-used live attenuated vaccines.

A group of viruses which has received significant attention from vaccine developers in the past is the group of non-segmented negative-strand RNA viruses (NNSV). These viruses have a very desirable safety profile since they contain an RNA genome and replicate only in the cytoplasm of host cells, excluding any possibility of integration into the host genome to cause insertional mutagenesis. Moreover, recombination events have not yet been observed (Bukreyev et al., J. Virol. 80:10293-10306, 2006). The NNSV comprise four families, of which members of the Rhabdoviridae (e.g., vesicular stomatitis virus (VSV) and rabies virus (RV)) and the Paramyxoviridae (e.g., Sendai virus (SeV) and human parainfluenza virus (hPIV)) have been preferentially used for the development of candidate viral vector vaccines (Schmidt et al., J. Virol. 75:4594-4603, 2001; Bukreyev et al., J. Virol. 80:10293-10306, 2006).

Using NNSV as vaccine backbones, various viral vaccine vector candidates have been developed. For example, a hPIV2/hPIV3 viral vaccine vector was produced by incorporation of HN and F proteins of human parainfluenza virus type 2 (hPIV2) having their cytoplasmic domains replaced with the corresponding ones of human parainfluenza virus type 3 (hPIV3) into a viral vector based on hPIV3 (Tao et al., J. Virol. 74:6448-6458, 2000). In addition, a bovine/human attenuated PIV3 vaccine vector was described, which expresses the F protein of hPIV3 in a bovine PIV3 (bPIV3) backbone (Haller et al., J. Virol. 74:11626-35, 2000). Further known is a bovine PIV3-based vaccine candidate expressing the F and NH proteins of human PIV3 and the full-length, native F protein of human RSV, which was found to confer protection from RSV infection in African green monkeys (Tang et al., J. Virol. 79:11198-11207, 2004).

Another candidate viral vector vaccine known in the art is based on a genome replication-deficient Sendai virus (SeV) (Wiegand et al., J. Virol. 81:13835-13844, 2007; WO 2006/084746 A1). This vector is still capable of expressing genes in vitro, as recently shown (Bossow et al., Open Virol. J. 6:73-81, 2012). In vivo safety of the replication-deficient SeV-based viral vaccine vector, however, concerning its replication-deficient nature and genetic stability, has still to be proven. In addition, the in vitro gene expression is, due to its replication-deficiency, reduced compared to that of replication-competent Sendai vectors (Bossow et al., Open Virol. J. 6:73-81, 2012). Therefore, it is a challenging task to recombinantly engineer a replication-deficient Sendai vector that efficiently expresses and displays selected immunogenic peptides or proteins to the immune system in a manner that results in the desired efficient humoral and/or cellular immune responses in vivo.

A well-known, but difficult to treat, pathogenic virus is the respiratory syncytial virus (RSV). RSV is a leading cause of serious respiratory diseases in young children and the elderly worldwide (Collins P. L. and Crowe J. E. Jr, Respiratory syncytial virus and metapneumovirus, in: Fields Virology, Eds. Knipe D. M. and Howley P., Philadelphia: Lippincott-Williams and Wilkins, Wolters Kluwer Business, 2007:1601-1646). RSV is also a major pathogen in chronic obstructive pulmonary disease (COPD) patients (Hacking, D. and Hull, J., J. Infect. 45:18-24, 2002). However, despite the significant RSV vaccine development efforts in recent times, there is still no vaccine available today against this pathogen.

Thus, there remains an urgent need for a safe RSV vaccine that is effective in the treatment of patients, in particular children and the elderly, suffering from RSV infections and RSV infection-related diseases.

### SUMMARY OF THE INVENTION

The present invention fulfills the need presented above by providing a genome replication-deficient Sendai virus (SeV) vector expressing a chimeric RSV/SeV F (fusion) protein or a RSV F protein comprising the ectodomain and the transmembrane domain (in the following referred to as “genome replication-deficient SeV vector of the present invention” or “rdSeV vector of the present invention”). The rdSeV vector of the present invention can be efficiently produced in high amounts and elicits strong humoral and cellular immune responses against RSV while at the same time being safe. It is therefore well-suited for use as a “semi-live” RSV vaccine, i.e. a vaccine that is exceptionally effective (like “live vaccines”) and yet particularly safe (like “dead vaccines”).

In a first aspect, the present invention provides a genome replication-deficient Sendai virus (SeV) vector comprising a nucleic acid that is modified in the phosphoprotein (P) gene to encode a mutant P protein lacking amino acids 2-77, wherein the nucleic acid further encodes a chimeric F protein comprising a respiratory syncytial virus (RSV) F ectodomain, or an immunogenic fragment or mutant thereof, a RSV F transmembrane domain, or a functional fragment or mutant thereof, and a SeV F cytoplasmic domain, or any fragment or mutant thereof (in the following “chimeric F protein” or “chimeric RSV/SeV protein”), or wherein the nucleic acid encodes an F protein comprising a RSV F ectodomain, or an immunogenic fragment or mutant thereof, and a RSV F transmembrane domain, or a functional fragment or mutant thereof (in the following “RSV F protein”).

In another aspect, the present invention provides a host cell comprising a genome replication-deficient Sendai virus (SeV) vector of the present invention, the nucleic acid of the genome replication-deficient SeV vector of the present invention or a complement thereof, and/or a DNA molecule encoding the nucleic acid of the genome replication-deficient SeV vector of the present invention or encoding a complement of the nucleic acid.

In a further aspect of the present invention, there is provided a method for producing the genome replication-deficient Sendai virus (SeV) vector of the present invention, comprising (i) culturing a host cell of the present invention, and (ii) collecting the genome replication-deficient SeV vector from the cell culture.

According to another aspect, the present invention provides a vaccine comprising the genome replication-deficient Sendai virus (SeV) vector of the present invention and one or more pharmaceutically acceptable carriers.

In yet another aspect, the present invention relates to the use of a genome replication-deficient Sendai virus (SeV) vector of the present invention in the treatment of RSV infections or RSV infection-related diseases in a mammal, particularly in a human subject, more particularly in a human infant or child, an elderly human, a human immunocompromised individual, a transplant recipient, or an individual suffering from a chronic disease.

Preferred embodiments of the present invention are set forth in the appended dependent claims.

### BRIEF DESCRIPTION OF THE DRAWINGS

The foregoing summary, as well as the following detailed description and examples, will be better understood when read in conjunction with the appended drawings.

FIG. 1 is a schematic representation showing the genome structure of a genome replication-deficient SeV vector of the present invention expressing a chimeric RSV/SeV F protein, designated as “rdSeV-F_{RSV/SeV}” vector. The ectodomain and transmembrane domains of SeV F were replaced by their corresponding RSV-derived counterparts resulting in the following chimeric F (“F_{chim2}”) protein: RSV ectodomain (“ecto”; amino acids 1-524 of RSV F), RSV transmembrane domain (“tm”; amino acids 525-550 of RSV F), and SeV cytoplasmic domain (“cyto”; amino acids 524-565 of SeV F). In the “Pₘᵤₜ” ORF, the first 76 amino acids were deleted (PΔ2-77) to obtain a replication-deficient vaccine vector.

FIG. 2 is a schematic representation showing the genome structure of a variant of the genome replication-deficient SeV vector of the present invention, designated as “rdSeV-F_{RSV/SeV}-ΔCT. This variant is identical to the rdSeV-F_{RSV/SeV} shown in FIG. 1 but lacks the entire cytoplasmic domain except for the N-terminal first two amino acids (amino acids 524-525 of SeV F). In the “Pₘᵤₜ” ORF, the first 76 amino acids were deleted (PΔ2-77) to obtain a replication-deficient vaccine vector.

FIG. 3 is a schematic representation showing the genome structure of a comparative genome replication-deficient SeV vector, designated “rdSeV-sF_{RSV}, which expresses a soluble F (sF) protein of RSV. The ORF of the RSV F ectodomain (amino acids 1-524 of RSV F) was inserted as an additional transcription unit (“sF_{RSV}”) downstream of the P gene. In the “Pₘᵤₜ” ORF, the first 76 amino acids were deleted (PΔ2-77) to obtain a replication-deficient vaccine vector.

FIG. 4 is a bar graph showing the production efficiency of the genome replication-deficient SeV vector of the present invention (rdSeV-F_{RSV/SeV}). The rdSeV-F_{RSV/SeV} vector was produced in VPN cells stably transfected with expression plasmids containing the genes coding for SeV P and N proteins. Different production runs of both vectors at different passaging levels (“P”) were performed in comparison (P1-1, P1-2, P2-1, P2-2, P3-1), and samples from the cell culture supernatants were taken at different time points during production, e.g. at day 8-11 (“d8-11”), day 11-12 (“d11-12”), and so forth. The vector titers (pfu/ml) of the samples taken were then determined.

FIG. 5 is a bar graph showing the production efficiency for rdSeV-F_{RSV/SeV} (black bars) and a variant thereof which lacks the entire cytoplasmic domain except for the N-terminal first two amino acids (designated as “rdSeV-F_{RSV/SeV}-ΔCT”) (white bars). The vector titers of cell culture supernatants in pfu/ml were determined at day 3 (“d2-3”), day 4 (“d3-4”), day 5 (“d4-5”), day 6 (“d5-6”) and day 7 (“d6-7”).

### DETAILED DESCRIPTION OF THE INVENTION

In accordance with the present invention, the genome replication-deficient SeV vector of the present invention provides a highly safe viral vector suitable for use as a vaccine against RSV infections and RSV infection-related diseases. Surprisingly, it was found that the genome replication-deficient SeV vector of the present invention can be highly efficiently produced in large amounts using cells that are qualified for human use. This allows for the cost-efficient production of the viral vaccine vector of the present invention, which is of utmost importance for a commercial vaccine. Furthermore, the genome replication-deficient SeV vector of the present invention can be produced in a simple and reproducible way and, due its small genome size, allows for constant and reliable sequence surveillance.

In a first aspect, the present invention provides a genome replication-deficient Sendai virus (SeV) vector. This vector comprises a nucleic acid that is modified in the phosphoprotein (P) gene to encode a mutant P protein lacking amino acids 2-77. The nucleic acid further encodes a specific chimeric RSV/SeV F protein or a specific RSV F protein comprising the RSV ectodomain and the RSV transmembrane domain. As used herein, a “Sendai virus vector” or “SeV vector” is an infectious virus comprising a viral genome. This is, the recombinant rdSeV vector of the present invention can be used for the infection of cells and cell lines, in particular for the infection of living animals including humans to induce immune responses against RSV infections.

Within the context of the present invention, the term “nucleic acid” is used in the broadest sense and encompasses single-stranded (ss) DNA, double-stranded (ds) DNA, cDNA, (−)-RNA, (+)-RNA, dsRNA and the like. However, when the nucleic acid is part of and included in the rdSeV vector of the present invention, the nucleic acid is negative-strand RNA ((−)-ssRNA). In this case, the nucleic acid corresponds typically to the genome of the rdSeV of the present invention. Further, the term “encoding”, as used herein, refers to the inherent property of a nucleic acid to serve as a template for the synthesis of another nucleic acid (e.g., mRNA, negative-strand RNA ((−)-ssRNA) or positive-strand RNA ((+)-ssRNA) and/or for the synthesis of oligo- or polypeptides (“proteins”). This is, a protein is “encoded” if transcription and translation results in the production of the protein in a cell or other biological system.

The SeV which serves as the backbone of the genome replication-deficient SeV vector of the present invention may be any known SeV strain. Suitable examples include, but are not limited to, the Sendai Fushimi strain (ATCC VR105), the Sendai Harris strain, the Sendai Cantell strain or the Sendai Z strain. The rdSeV of the present invention is further characterized by being replication-deficient (replication-defective). This is achieved by modifying the SeV backbone in the phosphoprotein (P) gene to delete the N-terminal 76 amino acids (PΔ2-77 of the P protein), as described previously (Bossow et al., Open Virol. J. 6:73-81, 2012; WO 2006/084746 A1). The resulting SeV/PΔ2-77 vector is replication-deficient, i.e. unable to synthesize new genomic templates in non-helper cell lines, but still transcription-competent, i.e. capable of primary transcription and gene expression, as shown previously (Bossow et al., Open Virol. J. 6:73-81, 2012).

Without being bound to any particular theory, it is believed that the deletion in the P protein, an essential component of the viral RNA-dependent RNA polymerase (vRdRp) carrying out both viral transcription and viral replication, uncouples the replication and transcription activities of the vRdRp. While this leads to a complete loss of the replication ability, the SeV/PΔ2-77 vector is still able to carry out primary transcription, including both early and late primary transcription. “Early primary” transcription refers to the first transcriptional events in an infected host cell, where the viral RNA genome is transcribed by the vRdRp molecules that were originally included in the SeV viral particles. “Late primary transcription” refers to the phase in which de novo protein synthesis begins and transcription is increasingly carried out by newly synthesised vRdRp.

In accordance with the present invention, the chimeric RSV/SeV protein encoded by the nucleic acid of the rdSeV vector of the present invention comprises (i) an ectodomain of a respiratory syncytial virus (RSV) F protein, or an immunogenic fragment or mutant thereof, (ii) a transmembrane domain of a RSV F protein, or a functional fragment or mutant thereof, and (iii) a cytoplasmic domain of a SeV F protein, or any fragment or mutant thereof. Likewise, the RSV F protein encoded by the nucleic acid of the rdSeV vector of the present invention comprises a RSV F ectodomain, or an immunogenic fragment or mutant thereof, and a RSV F transmembrane domain, or a functional fragment or mutant thereof.

The term “comprise”, as used herein, is intended to encompass both the open-ended term “include” and the closed term “consist (of)”. Thus, the nucleic acid of the rdSeV vector of the present invention may further encode other heterologous proteins or chimeric proteins resulting in, for example, a bivalent viral vector vaccine (e.g., directed against RSV and hPIV).

Within the present invention, the above-mentioned ectodomain and/or transmembrane domain of RSV may correspond to amino acids 1-524 and 525-550, respectively, of a RSV F protein. The SeV cytoplasmic domain may correspond to amino acids 524-565 of a SeV F protein. Thus, the chimeric RSV/SeV F protein may comprise 592 amino acids, of which amino acids 1-524 define the RSV ectodomain, amino acids 525-550 define the RSV transmembrane domain, and amino acids 551-592 define the SeV cytoplasmic domain. Deletion variants and mutants of this 592 amino acid chimeric RSV/SeV F protein are also within the scope of the present invention, wherein the “fragments” and “mutants” of the ectodomain, the transmembrane domain and the cytoplasmic domain are as defined below.

Preferably, the RSV ectodomain has the amino acid sequence shown in SEQ ID NO: 1 (ectodomain of RSV strain ATCC VR-26 (Long strain) F protein; GenBank accession no. AY911262, Translation AAX23994), or is an immunogenic fragment or mutant thereof. Preferably, the RSV transmembrane domain has the amino acid sequence shown in SEQ ID NO: 2 (transmembrane domain of RSV strain ATCC VR-26 (Long strain) F protein; GenBank accession no. AY911262, Translation AAX23994), or is a functional fragment or mutant thereof. Preferably, the SeV cytoplasmic domain has the amino acid sequences shown in SEQ ID NO: 3 (cytoplasmic domain of SeV strain Fushimi F protein; GenBank accession no. U06432, Translation AAC54271), or is any fragment or mutant thereof.

It is also preferred that the RSV ectodomain, the RSV transmembrane domain, and the SeV cytoplasmic domain are as defined above, except that the amino acid sequence of the RSV ectodomain shown in SEQ ID NO: 1 contains one or more, preferably all, point mutations selected from the group consisting of Glu66Gly, VaI76Glu, Asn80Lys, Thr101Ser and Ser211Asn, and/or the amino acid sequence of the SeV cytoplasmic domain shown in SEQ ID NO: 3 contains the single point mutation Gly34Arg. Particularly preferred, the chimeric RSV/SeV F protein has an amino acid sequence as defined by SEQ ID NOs: 1-3, or an amino acid sequence as defined by SEQ ID NOs: 1-3 containing all six point mutations indicated above. Similarly, the RSV F protein of the rdSeV vector of the present invention comprising a RSV ectodomain and a RSV transmembrane domain has most preferred an amino acid sequence as defined by SEQ ID NOs: 1 and 2, or an amino acid sequence as defined by SEQ ID NOs: 1 and 2 containing all five ectodomain point mutations indicated above, wherein fragments and mutants of said amino acid sequence are also encompassed by the present invention.

In the context of the present invention, the term “fragment” refers to a part of a polypeptide or protein domain generated by an amino-terminal and/or carboxy-terminal deletion. Preferably, the amino-terminal and/or carboxy-terminal deletion is no longer than 10 or 5 amino acids, particularly 1, 2 or 3 amino acids. The term “immunogenic”, as used herein, means a fragment or mutant of the RSV ectodomain that is still capable of eliciting a humoral and/or cellular immune response. Preferably, the immunogenic fragment or mutant, upon fusing it to the transmembrane domain having the amino acid sequence of SEQ ID NO: 2 and the cytoplasmic domain having the amino acid sequence of SEQ ID NO: 3, elicits a humoral and/or cellular immune response to a degree equal to or higher than 10%, 20%, 40%, 60% or 80% of that achieved by the full-length chimeric RSV/SeV F protein defined by the amino acid sequences of SEQ ID NOs: 1-3. The term “functional”, as used herein, refers to a transmembrane domain fragment or mutant that is functionally equivalent to the transmembrane domain, i.e. a fragment or mutant which is still capable of anchoring the chimeric RSV/SeV F protein and/or the RSV F protein of the rdSeV vector of the present invention to the membrane.

Within the present invention, the fragment of the SeV cytoplasmic domain (sometimes also referred to as “cytoplasmic tail”) can be as short as one amino acid or two to five amino acids. In this case, the respective chimeric RSV/SeV F protein may be referred to as “essentially lacking” a cytoplasmic domain. As demonstrated in the examples below, a variant of the chimeric RSV/SeV F protein that lacks the entire SeV cytoplasmic domain, except for the first and second N-terminal amino acids (e.g., amino acids 1 and 2 of SEQ ID NO: 3), was unexpectedly found to allow for a very high production efficiency, even higher than that achieved with the RSV/SeV F protein with the full-length SeV cytoplasmic domain. Therefore, since the cytoplasmic domain appears to be dispensable, chimeric RSV/SeV F proteins containing any fragments (parts) of the cytoplasmic domain or lacking the cytoplasmic domain are encompassed by the present invention.

The term “mutant”, as used herein, refers to a mutated polypeptide or protein domain, wherein the mutation is not restricted to a particular type of mutation. In particular, the mutation includes single-amino acid substitutions, deletions of one or multiple amino acids, including N-terminal, C-terminal and internal deletions, and insertions of one or multiple amino acids, including N-terminal, C-terminal and internal insertions, and combinations thereof. The number of inserted and/or deleted amino acids may be 1 to 10, particularly 1 to 5. In addition, 1 to 20, particularly 1 to 10, more particularly 1 to 5 amino acids may be mutated to (substituted by) another amino acid. Furthermore, the term “mutant” may also encompass mutated ectodomains, mutated transmembrane domains and mutated cytoplasmic domains, which are at least 75%, preferably at least 85%, more preferably at least 95%, and most preferably at least 97% identical to the amino acid sequence shown in SEQ ID NO: 1 (ectodomain of RSV strain ATCC VR-26 (Long strain) F protein), SEQ ID NO: 2 (transmembrane domain of RSV strain ATCC VR-26 (Long strain) F protein), and SEQ ID NO: 3 (cytoplasmic domain of SeV strain Fushimi F protein), respectively.

The SeV used as backbone and the SeV from which the cytoplasmic domain is derived may be the same or different. However, since the rdSeV of the present invention is generally constructed by replacing the SeV F ectodomain and transmembrane domain of the SeV backbone with the corresponding RSV F ectodomain (or immunogenic fragment or mutant thereof) and RSV F transmembrane domain (or functional fragment or mutant thereof), respectively, the SeV portion of the chimeric F protein is typically derived from the SeV that is used as backbone of the rdSeV vector of the present invention.

Suitable SeV strains for use as backbone and/or for construction of the chimeric RSV/SeV F protein include the Sendai Fushimi strain (ATCC VR-105), the Sendai Harris strain, the Sendai Cantell strain and the Sendai Z strain. Likewise, the RSV ectodomain may be derived from a RSV F protein from any recombinant or naturally-occurring RSV strain, preferable from a human SeV strain, such as A2, long, or B strains.

In one embodiment of the present invention, the nucleic acid of the genome replication-deficient SeV vector of the present invention encodes a soluble RSV F protein in addition to the chimeric RSV/SeV F protein or the RSV F protein comprising a RSV ectodomain and a RSV transmembrane domain. A “soluble F protein” within the meaning of the present invention is an F protein that lacks any stretch of amino acids which locates the F protein to the membrane and, in particular, refers to an F protein lacking both the transmembrane domain and the cytoplasmic domain. Thus, the soluble RSV F protein may be the ectodomain of a RSV F protein, or an immunogenic fragment or mutant thereof. The terms “fragment”, “immunogenic”, and “mutant” have the same meaning as defined above.

In a preferred embodiment, the soluble RSV F protein corresponds to amino acids 1-524 of a RSV F protein, or an immunogenic fragment or mutant thereof. In a particularly preferred embodiment, the soluble RSV F protein is the ectodomain of the RSV ATCC VR-26 strain (Long strain) F protein having the sequence shown in SEQ ID NO: 1, or an immunogenic fragment or mutant thereof.

If high expression of the heterologous gene encoding the soluble RSV F protein (in the following referred to as “sF transgene”) is desired, the sequence is preferably inserted into the 3′ region of the viral negative-strand RNA genome. The reason is that negative-strand RNA viruses like SeV most efficiently transcribe transcription units at the 3′ end of their negative-strand RNA genome. Transcript levels of genes further downstream gradually decrease, which is a phenomenon known as transcriptional gradient. This allows regulating the expression level of a heterologous transgene by inserting it at different sites in the viral genome. Within the present invention, it is preferred that the sF transgene is inserted between the P (i.e. Pₘᵤₜ; PΔ2-77) gene and the M gene.

The sF transgene may be inserted as a transcriptional cassette, comprising the nucleic acid sequence encoding the soluble RSV F protein operatively linked to a transcription start sequence, a transcriptional terminator and, preferably, translation signals. The sF transgene may also be operatively linked with an mRNA stabilizing element. For instance, a Woodchuck hepatitis virus post-trancriptional regulatory element (WPRE) may be inserted into the 3′UTR and/or 5′UTR region of the sF transgene in order to stabilize its mRNA and prolong its expression.

The incorporation of the sF transgene encoding a soluble RSV F protein allows for the presentation of RSV antigens in two different ways, namely as a chimeric RSV/SeV or RSV F surface protein displaying the RSV antigen as structural vector component being embedded in the viral envelope, and as a soluble RSV F protein. Thus, the additional expression of a soluble RSV F protein may assist in inducing a more effective and broad immune response involving the humoral and cellular arms of the immune system.

In another embodiment of the present invention, the nucleic acid of the rdSeV vector of the present invention does not encode a soluble RSV F protein, or any fragment or mutant thereof. Furthermore, within the context of the present invention, it is preferred that the rdSeV vector of the present invention does not encode a chimeric F protein, or fragment or mutant thereof, other than the chimeric RSV/SeV F protein, or fragment or mutant thereof, described in detail herein and, preferably, does also not encode a soluble RSV F protein, or any fragment or mutant thereof. Moreover, within the context of the present invention, it is preferred that the rdSeV vector of the present invention does not encode a membrane-bound F protein, or fragment or mutant thereof, other than the RSV F protein, or fragment or mutant thereof, described in detail herein and, preferably, does also not encode a soluble RSV F protein, or any fragment or mutant thereof. Also, the chimeric RSV/SeV F protein described in detail herein is preferably the sole heterologous protein expressed by the rdSeV of the present invention.

In addition to the modifications described above, the SeV vector of the present invention may include other modifications. In particular, it may be modified to carry additional mutations in one or more viral genes. For example, the rdSeV vector of the present invention may additionally contain one or more mutations in at least one of the genes coding for viral envelope proteins. These mutations can be introduced by recombinant techniques as known in the art and may lead to different effects, such as altered viral cell specificity.

The rdSeV vector of the present invention may also have one or more mutation in the C, W, and/or V open reading frames (ORFs) as a result of N-terminal deletions in the viral P protein, because the C, W, and V ORFs overlap with the N-terminal ORF of the P gene. Furthermore, the rdSeV vector of the present invention may additionally have a deletion of the alternative start codon ACG of the C′ gene. The C′ gene encodes a non-structural protein known to exhibit an anti-IFN response activity in infected cells. The deletion of the start codon of the C′ gene was found to result in increased expression levels of heterologous gene products in infected target cells.

In a second aspect, the present invention provides a host cell, which comprises a genome replication-deficient Sendai virus (SeV) vector of the present invention, a nucleic acid of the genome replication-deficient SeV vector of the present invention or a complement thereof, and/or a DNA molecule encoding the nucleic acid of the genome replication-deficient SeV vector of the present invention or encoding a complement of the nucleic acid.

A “complement” within the meaning of the present invention means a nucleotide sequence which is complementary to the sequence of the nucleic acid (i.e. an “antisense” nucleic acid). In this regard, it is noted that the nucleic acid generally corresponds to the genome of the rdSeV of the present invention. This is, the complement of the nucleic acid generally corresponds to the antigenome of the rdSeV of the present invention.

The host cell may be either a rescue cell (or “virus generating cell”) or a helper cell (or “amplification cell”). The rescue cell is used for the initial production of the rdSeV vector of the present invention. The rescue cell is typically a eukaryotic cell, particularly a mammalian cell, which usually expresses a heterologous DNA-dependent and/or RNA-dependent RNA polymerase, such as T7 RNA polymerase or the homologous cellular RNA polymerase II. The gene encoding the heterologous DNA-dependent RNA polymerase may be integrated into the rescue cell's genome or present in an expression plasmid.

The rescue cell must further express a functional SeV P protein as well as SeV N and L proteins so that the rdSeV vector of the present invention can be assembled. The expression of these viral proteins is typically achieved by transfecting the rescue cell with one or more expression plasmids carrying the respective P, N and L genes. A suitable rescue cell for use herein is a BSR-T7 cell, which contains the gene for the T7 RNA polymerase stably integrated in its genome, and which has been transfected with expression plasmids harbouring the genes for the SeV P, N and L proteins (Buchholz et al., J. Virol. 73:252-259, 1999).

In order to initially produce the rdSeV vector of the present invention, a DNA molecule encoding the nucleic acid of the rdSeV of the present invention or its antisense nucleic acid is transfected into a rescue cell. The cell transfection can be carried out in accordance with procedures known in the art, for example chemically with FuGENE 6 or FuGENE HD (Roche) reagents as described by the manufacturer, or by electroporation. The transfected DNA molecule is typically a plasmid carrying the cDNA of the nucleic acid of the rdSeV of the present invention. Since the DNA molecule is usually transcribed by a heterologous DNA-dependent RNA polymerase of the rescue cell, the DNA molecule preferably further includes a transcriptional signal, e.g. a T7 promoter, and a terminator sequence operatively linked with the viral genomic sequence. It may further include a ribozyme sequence at its 3′ end, which allows for cleavage of the transcript at the 3′ end of the viral sequence. The DNA molecule is further preferably suitable for propagation in a prokaryotic helper cell (e.g., Escherichia coli) and/or in a eukaryotic helper cell, in particular in a mammalian helper cell. After packaging the recombinant viral genome in the rescue cell and subsequent assembly of viral particles at the cell's surface, newly generated rdSeV vectors are released via budding from the cell and may be used for another round of infection of helper cells.

The helper cells (HPs) are used for amplifying the SeV vectors initially assembled in the rescue cell and are typically derived from mammalian cells, such as Vero cells or HEK-293 cells. These helper cells express the P protein and, optionally the N and/or L protein. The corresponding P, N and L genes may be integrated in the helper cells' genome or present in one or more expression plasmids. An exemplary suitable cell line is a cell line derived from HEK-293 cells, which constitutively express the SeV P protein (Willenbrink et al., J. Virol. 68:8413-8417, 1994). According to the present invention, the helper cells are preferably genetically modified to express the viral P and N proteins but not the viral L protein, since this P/N co-expression was surprisingly found to result in the highest virus production rates.

In a third aspect, the present invention provides a method for producing the genome replication-deficient Sendai virus (SeV) vector of the present invention, comprising the steps of:
(i) culturing a host cell of the present invention, and
(ii) collecting the genome replication-deficient SeV vector from the cell culture.

Methods for producing genome replication-deficient SeV vectors are known in the art and described in, for example, Wiegand et al., J. Virol. 81:13835-13844 (2007), Bossow et al., Open Virol. J. 6:73-81 (2012), and WO 2006/084746 A1. In the culturing step (i), the host cell is cultured in a suitable culture medium under conditions which permit genome replication and transcription so that the genome replication-deficient SeV of the present invention is formed. The medium used to culture the cells may be any conventional medium suitable for growing the host cells, such as DMEM (Invitrogen) supplemented with 10% heat-inactivated FCS. The host cell may be a rescue cell or a helper cell as defined above. In the collecting step (ii), the formed SeV vector of the present invention is recovered by methods known in the art.

In accordance with a preferred embodiment, the method for producing the genome replication-deficient Sendai virus (SeV) vector of the present invention comprises the following steps:
(a) introducing a DNA molecule into a first host cell, wherein the DNA molecule encodes the nucleic acid of the genome replication-deficient Sendai virus (SeV) vector of the present invention, or a complement thereof,
(b) culturing the first host cell to generate the genome replication-deficient SeV vector,
(c) collecting the genome replication-deficient SeV vector from the first cell culture,
(d) infecting a second host cell with the genome replication-deficient SeV vector obtained in step (c),
(e) culturing the second host cell to amplify the genome replication-deficient SeV vector,
(f) collecting the genome replication-deficient SeV vector from the second cell culture.

The first host cell is preferably a rescue cell (virus generating cell) as described above, and the second host cell is preferably a helper cell (amplification cell) as described above. The introduction of the DNA molecule into the first host cell in step (a) can be carried out by transfection methods known in the art. The culturing and collecting steps may be carried out as defined above.

In a fourth aspect, the present invention relates to a vaccine comprising the genome replication-deficient Sendai virus (SeV) vector of the present invention and one or more pharmaceutically acceptable carriers.

The term “vaccine”, as used herein, refers to an agent or composition containing an active component effective to induce a therapeutic degree of immunity in a subject against a certain pathogen or disease. The vaccine of the present invention is a “semi-live” vaccine, which refers to a vaccine that is not a live vaccine since it is replication-deficient, but is also not an inactivated (or killed) vaccine since it is still capable of primary transcription and gene expression. The semi-live vaccine of the present invention is exceptionally effective (like “live vaccines”) and yet particularly safe (like “dead vaccines”).

In the context of the present application, the dosage form of the vaccine of the present invention is not particularly limited and may be a solution, suspension, lyophilized material or any other form suitable for the intended use. For example, the vaccine may be in the form of a parenteral formulation, such as an aqueous or non-aqueous solution or dispersion for injection or infusion, or a formulation suited for topical or mucosal administration.

The vaccine generally includes an effective amount of the rdSeV of the present invention. Within the present invention, the term “effective amount” refers to the amount of a compound sufficient to effect beneficial or desired therapeutic results. A therapeutically effective amount can be administered in one or more administrations, applications or dosages and is not intended to be limited to a particular formulation or administration route.

Further included in the vaccine are one or more pharmaceutically acceptable carriers. The term “pharmaceutically acceptable”, as used herein, refers to those compounds or substances which are, within the scope of sound medical judgment, suitable for contact with the tissues of mammals, especially humans, without excessive toxicity, irritation, allergic response and other problem complications. The term “carrier”, as used herein, relates to diluents, adjuvants, excipients, vehicles or other compounds or substances needed, required or desired in a vaccine composition. Suitable carriers are especially those suited for parenteral, mucosal or topical administration, including sterile aqueous and non-aqueous solutions or dispersions for injection and infusion, as discussed in Remington: The Science and Practice of Pharmacy, 20th edition (2000).

In particular, the vaccine may comprise one or more adjuvants. The term “adjuvant”, as used herein, refers to an agent that enhances the immunogenicity of an antigen but is not necessarily immunogenic. Suitable adjuvants include, but are not limited to, 1018 ISS, aluminum salts, Amplivax®, AS 15, BCG, CP-870,893, CpG7909, CyaA, dSLIM, flagellin or TLR5 ligands derived from flagellin, FLT3 ligand, GM-CSF, IC30, IC31, Imiquimod (ALDARA®), resiquimod, ImuFact IMP321, interleukins such as IL-2, IL-13, IL-21, IFN-alpha or -beta, or pegylated derivatives thereof, IS Patch, ISS, ISCOMATRIX, ISCOMs, Juvlmmune, LipoVac, MALP-2 or natural or synthetic derivatives thereof, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK, and OspA.

In addition, the vaccine may include one or more additional active substances that are co-administered with the rdSeV vector of the present invention. In addition, the pharmaceutical composition may contain additional pharmaceutically acceptable substances, for example pharmaceutical acceptable excipients such as solubilizing agents, surfactants, tonicity modifiers and the like.

In a fifth aspect, the present invention relates to a genome replication-deficient Sendai virus (SeV) vector of the present invention for use in the treatment of RSV infection or RSV infection-related diseases in a mammal.

The term “treatment”, as used herein, is intended to refer to both therapeutic treatment and prophylactic treatment (or prevention) of a disease. In accordance with the present invention, “treatment” preferably means prophylactic treatment or prevention. A “treatment” within the meaning of the present invention generally involves the administration of an effective amount of the rdSeV vector of the present invention. Preferably, the rdSeV of the present invention is administered in the form of a vaccine composition as described herein.

The mammal to be treated is preferably a human subject. Particularly important target groups are human infants and children, in particular a human infant born prematurely or a human infant at risk of hospitalization for a RSV infection.

Other important target groups include elderly humans, human immunocompromised individuals, transplant recipients, especially organ transplant recipients, and individuals suffering from a chronic disease. The chronic disease may be, for example, cancer, chronic hepatitis, ischemic cardiopathy, chronic renal failure, chronic respiratory diseases (e.g., asthma, obstructive pulmonary disease (COPD), pulmonary hypertension), chronic graft-versus-host disease (GVHD), and autoimmune diseases (e.g., lupus erythematosus, ulcerative colitis, inflammatory bowel diseases (IBD), Crohn's disease).

The RSV infections include all type of respiratory tract infections associated with RSV. The RSV infection-related diseases are preferably selected from the group consisting of otitis media, bronchilitis, eosinophilia, pneumonia, asthma, and chronic obstructive pulmonary disease (COPD).

Suitable administration routes include, but are not limited to, parenteral, mucosal and topical administration. The parenteral administration may be by subcutaneous, intravenous, intraperitoneal or intramuscular injection. Mucosal administration may include administration to an airway surface, such as by droplet administration to a nasal surface or sublingual administration, or by inhalation administration of aerosolized particles to a nasal surface or the surfaces of other airway passages.

As demonstrated in the examples below, the genome replication-deficient SeV vector of the present invention effectively elicits mucosal immune responses when administered intranasally. Therefore, although the genome replication-deficient SeV vector or vaccine of the present invention may be administered via any traditional route, it is preferably administered mucosally, for example via the nasal or oral (intragastric) routes. Particularly preferred is the intranasal administration.

The administration regimen is not particularly limited and includes, for example, daily, bi-weekly, monthly, once every other month, once every third, sixth or ninth month and once-a-year or single application administration schemes. The therapeutically effective dose of the virus vector that is administered to the patient depends on the mode of application, the type of disease, the patient's weight, age, sex and state of health, and the like. Administration can be single or multiple, as required. The vaccine of the present invention may also be co-administered with antigens from other pathogens as a multivalent vaccine.

The present invention will now be further illustrated by the following, non-limiting examples.

### EXAMPLES

In the following examples, the genetic stability, safety and production efficiency of a replication-deficient Sendai virus vector of the present invention (in the following referred to as “rdSeV-F_{RSV/SeV}” vector) were evaluated. The results show that the rdSeV-F_{RSV/SeV} vector is safe and can be efficiently produced in high amounts. Thus, the rdSeV vector of the present invention is a promising viral vector vaccine candidate against RSV infections and RSV infection-related diseases.

### Materials and Methods

The following materials and methods were used in Examples 1-5.

Cells and Viruses:

Vero (ATCC CCL-81), HEp-2 (ATCC CCL-23) and P815 cells (ATCC TIB-64) from the American Type Culture Collection (Rockville, Md., USA) were maintained in Eagle minimal essential medium or RPMI (Invitrogen, Milan, Italy) supplemented with 5% heat-inactivated foetal bovine serum (FBS; Invitrogen), 100 μg/ml streptomycin and 100 U/ml penicillin. The helper cell line “P-HC” (“amplification cells”) is derived from Vero cells expressing SeV phosphoprotein (protein P) (Wiegand et al., J. Virol. 81:13835-13844, 2007), and the helper cell line “VPN” is derived from Vero cells expressing the plasmid-encoded SeV phosphoprotein (protein P) and nucleoprotein (protein N). BSR-T7 cells (“rescue cells”) (Buchholz et al., J. Virol. 73:251-259, 1999) were kindly provided by Klaus-K. Conzelmann (Munich). RSV type A (Long strain, ATCC VR-26) was cultured on HEp-2 cells at 37° C. All vaccine candidates (rdSeV-F_{RSV/SeV}, rdSeV-F_{RSV/SeV}-ΔCT, rdSeV-sF_{RSV}) based on recombinant SeV vectors derived from Sendai virus strain D52 (ATCC VR-105) were cultured at 33° C.

Genomic Vector Design:

For the construction of a virus vector of the present invention, plasmids containing the cDNA of the RSV or SeV F gene, respectively, were used as templates for the construction of a chimeric RSV/SeV F ORF by an overlapping PCR technique (Horton et al., Gene 77:61-68, 1989). Via specific primer design, non-overlapping regions at the 3′- and 5′-ends containing specific sequences for the restriction enzymes SalI and XhoI, were introduced. The sequence-verified chimeric ORF was inserted into a subgenomic plasmid construct, comprising the Sendai virus genome from the SanDI restriction site within the P gene of the wild-type genome (genomic nucleotide position 2714) until the SanDI restriction site within the L gene (genomic nucleotide position 9131). This genomic fragment was modified in a way that the F ORF was flanked by the restriction sites for SalI and XhoI. After insertion of the chimeric F ORF into the intermediate cloning vector the full length genome of rdSeV-F_{RSV/SeV} was created via transfer of the SanDI fragment from the cloning vector into the previously prepared, full length construct of rdSeV. The resulting recombinant SeV genome, following the “rule of six” (Calain et al., J. Virol. 67:4822-4830, 1993), was designated “rdSeV-F_{RSV/SeV}” (replication-deficient SeV encoding a chimeric RSV/SeV F protein), and was confirmed by restriction analysis and sequencing.

The rdSeV-sF_{RSV} vector expressing a soluble RSV F protein was generated by transferring the subgenomic EcoRI fragment from the recombinant Sendai vector encoding the soluble form of the RSV F protein as additional transgene between the P and the M gene, as described by Voges et al. (Voges et al., Cell. Immunol. 247:85-94, 2007), into a replication-deficient Sendai vector as described in WO 2006/084746 A1. The resulting recombinant SeV genome, following the “rule of six” (Calain et al., J. Virol. 67:4822-4830, 1993), was designated “rdSeV-sF_{RSV}” (replication-deficient SeV vector expressing RSV soluble F protein), and was confirmed by restriction analysis and sequencing.

Virus Rescue, Propagation and Titration:

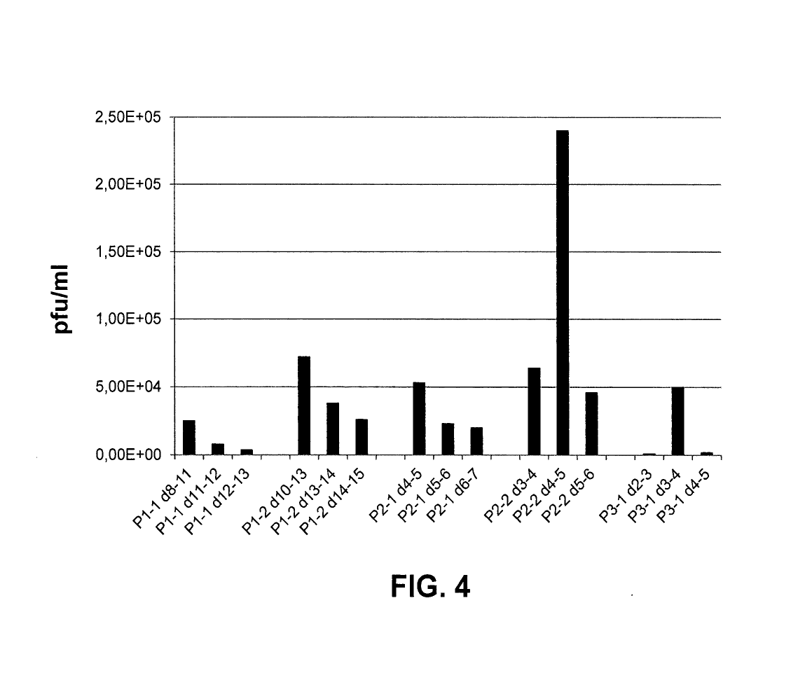

Recombinant viruses were recovered from transfected BSR-T7 cells as described in Wiegand et al., J. Virol. 81:13835-13844, 2007 with slight modifications. FuGENE6 (Roche) was used as transfection reagent at 2.0 μl/μg DNA. Replication-deficient SeV virus was harvested from the supernatant and amplified in a helper cell line stably expressing the SeV P protein (“P-HC”). This P-HC line was used in all experiments, except for the experiments in relation to virus production efficiency (see FIG. 4), where the vaccine vector rdSeV-F_{RSV/SeV} was produced in a VPN helper cell line stably expressing the Sendai virus P and N proteins (Wiegand et al., J. Virol. 81:13835-13844, 2007). Viruses were titrated as previously described (Wiegand et al., J. Virol. 81:13835-13844, 2007) and titers were given as cell infectious units per millilitre (ciu/ml) (equivalent to fluorescent plaque forming units). The integrity of the different SeV vectors was confirmed by RT-PCR and sequencing.

Western Blot Analysis:

Extracts from Vero cells, mock infected or infected with PIV3, RSV or rdPIRV, were collected and separated by SDS-PAGE. After blotting on a nitrocellulose membrane proteins were detected with mouse monoclonal antibodies against PIV3 HN and F proteins (Chemicon, Milan, Italy) and a goat anti-RSV antibody (Meridian Life Science, Saco, Me.).

### Example 1

### Generation of an Inventive Replication-Deficient SeV Vector

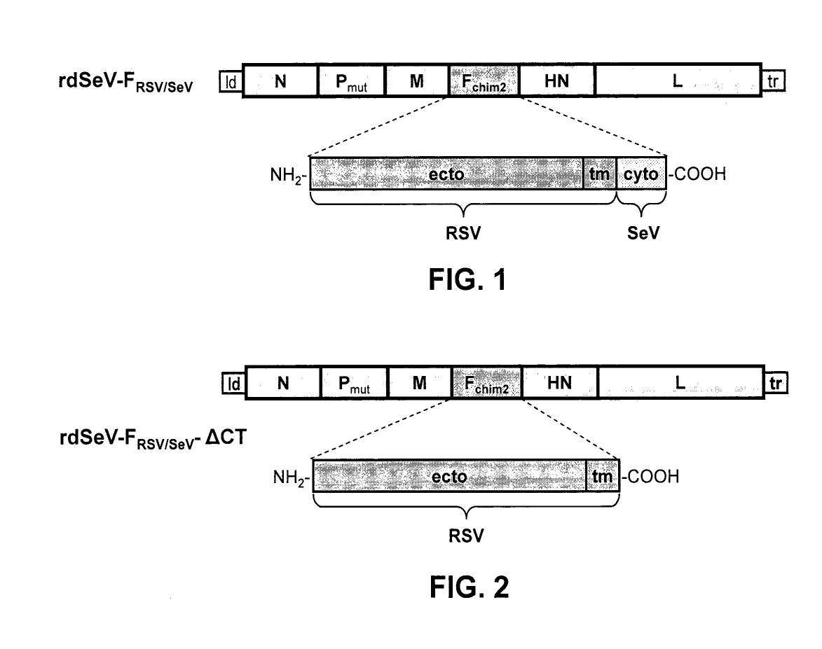

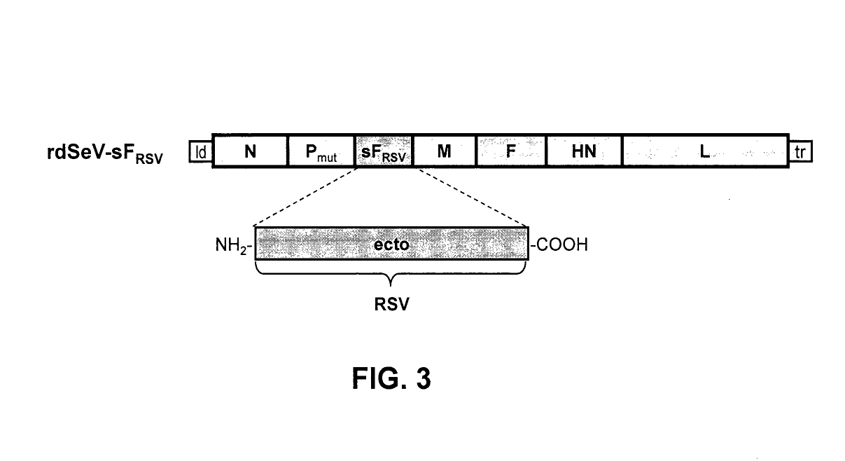

Using reverse genetic techniques, a SeV vaccine vector against human RSV, named “rdSeV-FR_{RSV/SeV}” (replication-deficient SeV vector expressing chimeric RSV/SeV F protein), was constructed. The SeV F ORF, except for the cytoplasmic domain, was replaced by its RSV counterpart to give a chimeric RSV/SeV F surface protein (FIG. 1). In addition, in order to develop a safe vaccine vector, the SeV backbone was modified in the phosphoprotein (P) gene by deleting the N-terminal 76 amino acids (PΔ2-77). As shown previously, a SeV vector with the deletion PΔ2-77 is unable to synthesize new genomic templates in non-helper cell lines, but it still capable of primary transcription and gene expression (Bossow et al., Open Virol. J. 6:73-81, 2012). The rdSeV-F_{RSV/SeV} could be rescued successfully from cDNA and amplified using the helper cell line “P-HC”.

### Example 2

### Genetic Stability of Replication-Deficient SeV Vectors

In this example, the genetic stability of genome replication-deficient SeV vectors was evaluated using a specific replication-deficient SeV construct referred to as “rdPIRV” (replication-deficient PIV3/RSV SeV vector). Although this construct is not within the scope of the appended claims, the results obtained for this construct with regard to stability are also considered valid for the genome replication-deficient SeV vector of the present invention.

The rdPIRV vector is genetically engineered to express a soluble RSV F protein as well as chimeric RSV/SeV F and HN surface proteins using techniques described above and/or known in the art. In brief, the RSV F ectodomain coding sequence was inserted as an additional transcription unit being expressed as soluble protein (sF) as successfully employed previously (Voges et al., Cell. Immunol. 247:85-94, 2007). The SeV F and HN ORFs were replaced, except for the cytoplasmic and transmembrane domains, by their PIV3 counterparts. Furthermore, in order to develop a safe vaccine vector, the SeV backbone was modified in the phosphoprotein (P) gene by deleting the N-terminal 76 amino acids (PΔ2-77).

The rdPIRV could be rescued successfully from cDNA and amplified using a helper cell line. This vector was unable to synthesize new genomic templates in non-helper cell lines, but it was still capable of primary transcription and gene expression, as demonstrated by Western Blot analysis of PIV3 F and HN and RSV sF protein expression (data not shown). Further, sequence analyses after ten consecutive passages revealed no mutations.

These results confirm the structural integrity and sequence stability of the replication-deficient SeV/PΔ2-77 vaccine vector and, thus, of the replication-deficient SeV vector of the present invention.

### Example 3

### Safety of Replication-Deficient SeV Vectors

In addition, studies regarding the safety of replication-deficient SeV vectors, in particular on replication-deficiency and biodistribution to different tissues in vivo, were performed with the rdPIRV vector described in Example 2. Again, the results obtained for the rdPIRV vector with regard to safety are considered to equally apply to the genome replication-deficient SeV vector of the present invention.

Two groups of BALB/C mice (n=4) were inoculated intranasally (i.n.) with 1×10⁵ ciu of rdPIRV or a modified replication-competent SeV (SeV-E wt) expressing the EGFP (Enhanced Green Fluorescent Protein) to facilitate its detection. After three days, mice were sacrificed and lungs and blood samples were collected. Virus present in tissue homogenates and blood was quantified by counting EGFP-positive foci on cell culture (detection limit: 20 ciu per lung, per spleen or per 500 μl blood).

No viral particles of rdPIRV could be detected in any animal tissue examined. Only when SeV-E wt was used, viral particles could be detected in the lungs (up to 3.2×10⁴ ciu per lung), but not in blood (data not shown). In addition, lung homogenates drawn from rdPIRV-immunized mice were overlayed onto Vero cells to verify the absence of any replicating recombinant SeV. No virus could be detected, confirming that this vaccine vector was replication-deficient in vivo (data not shown). No animal developed any signs of pain or weight loss.

Taken together, these data demonstrate that: (i) deletion of amino acids 2-77 in the P gene disables the vector from producing progeny genomes in vivo; (ii) replication competent SeV spreading is limited to the respiratory tract. These results also apply to the replication-deficient SeV vector of the present invention, which is therefore considered particularly safe for administration to humans.

### Example 4

### Production Efficiency

Production efficiency of commercial vaccines has a huge impact on the market potential of such products. Therefore, production efficiency of the genome replication-deficient SeV vector of the present invention (rdSeV-FR_{RSV/SeV} vector) was assessed and compared with that of the variant rdSeV-F_{RSV/SeV}-ΔCT lacking the cytoplasmic domain.

In a first study, VPN helper cells stably transfected with the genes coding for the SeV P and N proteins were infected with the inventive rdSeV-F_{RSV/SeV} vector. Different passages of the vector (P1, P2, P3) were analyzed. For passage P1 and P2 even two separate production runs were performed (P1-1, P1-2, P2-1, P2-2). The samples taken at different time points (e.g., at day 8-11 (“d8-11”), day 11-12 (“d11-12”), and so forth) from the cell culture supernatants were analyzed for their vector titers.

As can be seen from FIG. 4, the virus titers are remarkably high at all passaging levels and production runs, particularly during passage P2. Overall, these results demonstrate unexpectedly high production efficiency due to the presence of two surface proteins (F and HN) from two different viruses at the same time. This finding was surprising since a strong interference during the processes of attachment fusion and budding was expected.

In a second study, the production efficiency of rdSeV-F_{RSV/SeV} was compared with a variant thereof coding for an F protein essentially lacking its cytoplasmic tail (“rdSeV-F_{RSV/SeV}-ΔCT”) (see FIG. 2). This variant was spontaneously generated during sequential passaging of rdSeV-F_{RSV/SeV} on the helper cell line “P-HC”. Subsequent sequence analysis of the produced vector particles revealed that a nonsense mutation in the K553 (Lys-553) codon of the F gene resulted in a premature stop codon. As a consequence, only the first two amino acids of the SeV F cytoplasmic domain (i.e. amino acids 524 and 525) are retained in this variant, which therefore (essentially) lacks its cytoplasmic tail.

During subsequent passaging of the spontaneously generated variant without cytoplasmic tail in cell culture, it was observed that the ratio of the deletion variant to non-mutated virus (rdSeV-F_{RSV/SeV}) increased. Based on this unexpected observation, it was subsequently confirmed by means of comparative production rounds in cell cultures of non-mutated virus (i.e. rdSeV-F_{RSV/SeV}) and mutated variant (i.e. rdSeV-F_{RSV/SeV}-ΔCT) that the mutant virus could be amplified to a significantly higher titer. In brief, cells were infected with the same MOI of 0.1 and cultured for five days. At different times points, i.e. at day 3 (“d2-3”), day 4 (“d3-4”), day 5 (“d4-5”), day 6 (“d5-6”) and day 7 (“d6-7”), the vector titers of cell culture supernatants were determined.

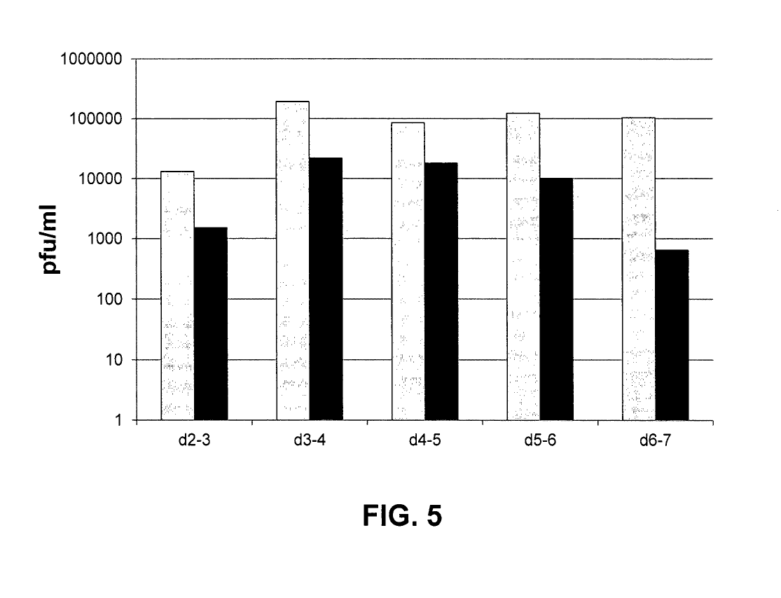

As can be seen from FIG. 5, as early as at day 3 the titer of rdSeV-F_{RSV/SeV}-ACT was 5-fold higher than that of rdSeV-F_{RSV/SeV}. At day 4 and day 5, respectively, the titer of rdSeV-F_{RSV/SeV}-ΔCT was 5-fold to 10-fold higher than that of rdSeV-F_{RSV/SeV}, and the titer at day 6 and day 7 was even more than 10-fold higher. This finding was altogether unexpected since the prior art teaches that the cytoplasmic tail of the SeV F protein plays a critical role in virus assembly (see Stone, R. and Takimoto, T., PLoS ONE 8(4): e61281. doi:10.1371/journal.pone.0061281, 2013). Thus, if anything, the skilled person would have expected to obtain decreased production efficiency. However, the deletion mutant rdSeV-F_{RSV/SeV}-ΔCT was surprisingly found to exhibit excellent production efficiency, even much better than that of rdSeV-F_{RSV/SeV} expressing the full-length chimeric RSV/SeV F protein.

Overall, the above results show that the rdSeV vector of the present invention has a superior safety profile and allows to achieve a surprisingly high production efficiency. High production efficiency is a very important and desirable feature of a viral vector with regard to its commercialization as a vaccine. Thus, the rdSeV vector of the present invention is a very promising vaccine candidate against RSV.

We claim:

1. A genome replication-deficient Sendai virus (SeV) vector comprising a nucleic acid that is modified in the phosphoprotein (P) gene to encode a mutant P protein lacking amino acids 2-77, wherein the nucleic acid further encodes a chimeric F protein comprising a respiratory syncytial virus (RSV) F ectodomain, or an immunogenic fragment or mutant thereof, a RSV F transmembrane domain, or a functional fragment or mutant thereof, and a SeV F cytoplasmic domain, or any fragment or mutant thereof, or wherein the nucleic acid further encodes a F protein comprising a RSV F ectodomain, or an immunogenic fragment or mutant thereof, and a RSV F transmembrane domain, or a functional fragment or mutant thereof.

2. The genome replication-deficient SeV vector of claim 1, wherein the RSV ectodomain corresponds to amino acids 1-524 of a RSV F protein and/or the RSV transmembrane domain corresponds to amino acids 525-550 of a RSV F protein and/or the SeV cytoplasmic domain corresponds to amino acids 524-565 of a SeV F protein.

3. The genome replication-deficient SeV vector of claim 1, wherein the chimeric F protein essentially lacks a cytoplasmic domain.

4. The genome replication-deficient SeV vector of claim 1, wherein the nucleic acid further encodes a soluble RSV F protein, or an immunogenic fragment or mutant thereof.

5. The genome replication-deficient SeV vector of claim 4, wherein the soluble RSV F protein is the ectodomain of a RSV F protein, or an immunogenic fragment or mutant thereof.

6. The genome replication-deficient SeV vector of claim 1, wherein the nucleic acid does not encode a soluble RSV F protein, or an immunogenic fragment or mutant thereof.

7. A host cell comprising a genome replication-deficient Sendai virus (SeV) vector according to claim 1, the nucleic acid of the genome replication-deficient SeV vector according to claim 1 or a complement thereof, and/or a DNA molecule encoding the nucleic acid of the genome replication-deficient SeV vector according to claim 1 or encoding a complement of the nucleic acid.

8. A method for producing the genome replication-deficient Sendai virus (SeV) vector according to claim 1, comprising:
(i) culturing a host cell according to claim 7, and
(ii) collecting the genome replication-deficient SeV vector from the cell culture.

9. A vaccine comprising the genome replication-deficient Sendai virus (SeV) vector according to claim 1 and one or more pharmaceutically acceptable carriers.

10. The vaccine of claim 9, further comprising an adjuvant.

11. A method for the treatment of RSV infections or RSV infection-related diseases in a mammal the method comprising administering to said mammal a genome replication-deficient Sendai virus (SeV) vector comprising a nucleic acid that is modified in the phosphoprotein (P) gene to encode a mutant P protein lacking amino acids 2-77, wherein the nucleic acid further encodes a chimeric F protein comprising a respiratory syncytial virus (RSV) F ectodomain, or an immunogenic fragment or mutant thereof, a RSV F transmembrane domain, or a functional fragment or mutant thereof, and a SeV F cytoplasmic domain, or any fragment or mutant thereof, or wherein the nucleic acid further encodes a F protein comprising a RSV F ectodomain, or an immunogenic fragment or mutant thereof, and a RSV F transmembrane domain, or a functional fragment or mutant thereof.

12. The method according to claim 11, wherein the mammal is a human subject.

13. The method according to claim 11, wherein the human subject is a human infant or child, including a human infant born prematurely or a human infant at risk of hospitalization for a RSV infection, an elderly human, a human immunocompromised individual, a transplant recipient, or an individual suffering from a chronic disease.

14. The method according to claim 11, wherein the vaccine is administered parenterally, topically or mucosally.

15. The method according to claim 14, wherein the parenteral administration is by subcutaneous, intravenous, intraperitoneal or intramuscular injection.


SEQUENCE LISTING (3 sequences)

SEQ ID NO: 1 - protein, 524 aa, Artificial Sequence
  “Ectodomain of RSV strain ATCC VR-26 F protein”
  Ectodomain of RSV strain ATCC VR-26 F protein
MELPILKANAITTILAAVTFCFASSQNITEEFYQSTCSAVSKGYLSALRTGWYTSVITIELSNIKENKCNGTDAKVKLINQELDKYKNAVTELQLLMQSTTAANNRARRELPRFMNYTLNNTKKTNVTLSKKRKRRFLGFLLGVGSAIASGIAVSKVLHLEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHHVNAGKSTTN

SEQ ID NO: 2 - protein, 26 aa, Artificial Sequence
  “Transmembrane domain of RSV strain ATVV VR-26 F protein”
  Transmembrane domain of RSV strain ATVV VR-26 F protein
IMITTIIIVIIVILLSLIAVGLLLYC

SEQ ID NO: 3 - protein, 42 aa, Artificial Sequence
  “Cytoplasmic domain of SeV strain Fushimi (V52) F protein”
  Cytoplasmic domain of SeV strain Fushimi (V52) F protein
RLKRSMLMGNPDDRIPRDTYTLEPKIRHMYTNGGFDAMAEKR